(19) United States
(12) Patent Application Publication
(10) Pub. No.: US 2024/0219334 A1
(43) Pub. Date: Jul. 4, 2024

(54) METHOD OF MANUFACTURING A NON-POLAR VOLATILE ORGANIC CHEMICAL SENSOR

(71) Applicant: Nanyang Technological University, Singapore (SG)

(72) Inventors: Jing Hao CIOU, Singapore (SG); Pooi See LEE, Singapore (SG)

(21) Appl. No.: 18/556,802

(22) PCT Filed: May 17, 2022
(86) PCT No.: PCT/SG2022/050324

§ 371 Date: Oct. 23, 2023

(30) Foreign Application Priority Data
May 17, 2021 (SG) 10202105123P

(51) Int. Cl. G01N 27/12; G01N 33/00

(52) U.S. Cl. CPC G01N 27/12; G01N 33/0027

(57) ABSTRACT

Disclosed herein is a method of manufacturing a non-polar volatile organic chemical sensor, the method requiring the steps of providing a composite material comprising a substrate coated by an activated Ti3C2 MXene layer, which activated Ti3C2 MXene layer has a surface and forming a hydrophobic silane layer on the surface of the activated Ti3C2 MXene layer to provide the non-polar volatile organic chemical sensor, where the hydrophobic silane layer is formed by gaseous phase silanization using a monomeric hydrophobic silane compound. Also disclosed herein is a non-polar volatile organic chemical sensor and its use to detect non-polar volatile organic compounds.

### FIELD OF INVENTION

The current invention relates to a method of making Ti₃C₂ MXenes that are useful in the detection of volatile organic compounds from plants. The invention also relates to their use in sensors.

### BACKGROUND

The listing or discussion of a prior-published document in this specification should not necessarily be taken as an acknowledgement that the document is part of the state of the art or is common general knowledge.

Due to the hydrophilic nature of pristine Ti₃C₂ MXene, high sensing response towards polar volatile organic compounds (VOCs) or gas, especially NH₃ gas (E. Lee et al., ACS Appl. Mater. Interfaces 2017, 9, 37184-37190; M. Wu et al., ACS Sens. 2019, 4, 2763-2770; and B. Xiao et al., Sens. Actuators B Chem. 2016, 235, 103-109), has been reported. However, there is still a lack of reports investigating Ti₃C₂ MXene for the sensing of non-polar VOCs. The only one work related to non-polar VOC sensing by Ti₃C₂ MXene was reported by Shuvo et al. (S. N. Shuvo et al., ACS Sens. 2020, 5, 2915-2924). The sulfur-doped Ti₃C₂ MXene presents the highest sensing response for analytes such as hexyl acetate, hexane and ethanol.

Apart from the common non-polar VOCs like toluene, there is another large group of hydrocarbons called terpenes. Terpenes contain diverse types of non-polar molecules. Interestingly, terpenes often have a certain odour, which deters an herbivore or attracts the predator of herbivores. In addition, the release of this kind of “signal” VOCs was also found to induce interplant communication for the defensive response to herbivores. Thus, the ability to detect these inter-plant communication signals would be a promising technique in farmland management and help to alarm the possible spread of plant disease or pest.

Therefore, there exists a need to discover new derivatives of Ti₃C₂ MXenes for the detection of plant VOCs.

### SUMMARY OF INVENTION

Aspects and embodiments of the current invention will now be discussed by reference to the following numbered clauses.
1. A method of manufacturing a non-polar volatile organic chemical sensor, the method comprising the steps of:
(a) providing a composite material comprising a substrate coated by an activated Ti₃C₂ MXene layer, which activated Ti₃C₂ MXene layer has a surface; and
(b) forming a hydrophobic silane layer on the surface of the activated Ti₃C₂ MXene layer to provide the non-polar volatile organic chemical sensor, wherein the hydrophobic silane layer is formed by gaseous phase silanization using a monomeric hydrophobic silane compound.
2. The method according to Clause 1, wherein the monomeric hydrophobic silane compound is selected from one or more of the group consisting of (3-aminopropyl)trimethoxysilane (APTMS), trimethoxy(octyl)silane (TEOS), trimethoxy(propyl)silane, and trimethoxymethylsilane (TEMS).
3. The method according to Clause 2, wherein the monomeric hydrophobic silane compound is trimethoxy(propyl)silane.
4. The method according to any one of the preceding clauses, wherein the hydrophobic silane layer has a thickness of from 10 to 14 nm.
5. The method according to any one of the preceding clauses, wherein the gaseous phase silanization is conducted at from 80 to 150° C., such as about 100° C. and a pressure of from 0.1 to 0.3 mbar, such as about 0.16 mbar.
6. The method according to any one of the preceding clauses, wherein the gaseous phase silanization used a flow of an inert gas at a flow rate of from 50 to 100 sccm, such as about 65 sccm, optionally wherein the inert gas is argon.
7. The method according to any one of the preceding clauses, wherein the composite material provided in step (a) of Clause 1 further comprises at least two electrodes laid on the surface of the activated Ti₃C₂ MXene layer.
8. The method according to Clause 7, wherein the electrodes are formed by thermal evaporation of an electrode material onto the surface of the activated Ti₃C₂ MXene layer, optionally wherein:
(i) the electrode material is gold; and/or
(ii) the at least two electrodes each have a thickness of from 75 to 150 nm, such as about 100 nm.
9. The method according to any one of the preceding clauses, wherein the activated Ti₃C₂ MXene layer is formed by the steps of:
(ai) providing a composite material comprising a substrate coated by an unactivated Ti₃C₂ MXene layer; and
(aii) activating the Ti₃C₂ MXene layer through a surface activation process.
10. The method according to Clause 9, wherein the surface activation process is plasma activation, optionally wherein the plasma activation is O₂ plasma activation.
11. A non-polar volatile organic chemical sensor comprising:
   a substrate having a substrate surface;
   a Ti₃C₂ MXene layer coated on the substrate surface which Ti₃C₂ MXene layer has a Ti₃C₂ MXene layer surface;
   at least two electrodes on the Ti₃C₂ MXene layer surface; and
   a hydrophobic silane layer on the Ti₃C₂ MXene layer surface, wherein the hydrophobic silane layer has a thickness of from 10 to 14 nm.
12. The sensor according to Clause 11, wherein the hydrophobic silane layer is formed from a monomeric hydrophobic silane compound that is selected from one or more of the group consisting of (3-aminopropyl)trimethoxysilane (APTMS), trimethoxy(octyl)silane (TEOS), trimethoxy(propyl)silane, and trimethoxymethylsilane (TEMS).
13. The sensor according to Clause 12, wherein the monomeric hydrophobic silane compound is trimethoxy(propyl)silane (TMPS).
14. The sensor according to any one of Clauses 11 to 13, wherein:
(I) the electrode material is gold; and/or
(II) the at least two electrodes each have a thickness of from 75 to 150 nm, such as about 100 nm.
15. The sensor according to any one of Clauses 11 to 14, wherein the non-polar volatile organic chemical sensor is capable of detecting an analyte present in an environment in a concentration of about 50 ppm.
16. A method of detecting an analyte comprising the steps of:
(bi) exposing a non-polar volatile organic chemical sensor as described in any one of Clauses 11 to 14 to an environment where the analyte is suspected to be present for a period of time; and
(bii) subsequently measuring the resistance of the volatile organic chemical sensor to determine the presence or absence of the analyte in said environment.
17. The method according to Clause 16, wherein the analyte is a volatile organic compound.
18. The method according to Clause 17, wherein the volatile organic compound is selected from one or more of α-pinene, 1-hexanol, a terpinol, and phenethyl alcohol, optionally wherein the volatile organic compound is α-pinene.
19. The method according to any one of Clauses 16 to 18 wherein the non-polar volatile organic chemical sensor is capable of detecting an analyte present in the environment in a concentration of about 50 ppm.

### DRAWINGS

FIG. 1 depicts the schematic illustration of the fabrication procedures of Ti₃C₂ MXene VOC sensor.

FIG. 2 depicts the X-ray photoelectron spectroscopy (XPS) wide range survey of (a) the pristine Ti₃C₂ MXene and (b) trimethoxy(propyl)silane (TMPS)-modified Ti₃C₂ MXene.

FIG. 3 depicts the XPS spectrum of TMPS-modified Ti₃C₂ MXene (tmpsMX) after gaseous phase silanization with measured core level of (a) C1 s, (b) O1 s and (c) Si2p and the measured core level of pristine Ti₃C₂ MXene (pMX) at (d) C1 s, (e) O1 s and (f) Si2p.

FIG. 4 depicts the I-V curve measured from pristine Ti₃C₂ MXene (pMX) VOC sensor, and TMPS-modified Ti₃C₂ MXene (tmps-MX) VOC sensor via two-terminal method.

FIG. 5 depicts (a) the dynamic gas sensing response of pristine Ti₃C₂ MXene (pMX) VOC sensor with the zoom in view inset from 0 s to 1400 s. The orange blocks highlight the time period with the α-Pinene purging in the testing chamber with the purging time: 50 ppm: 300 sec, 100 ppm: 300 sec, 500 ppm: 300 sec, 1000 ppm: 650 sec; and 2500 ppm: 800 sec; and (b) the dynamic gas sensing response of TMPS-modified Ti₃C₂ MXene (tmps-MX) VOC sensor with zoom in view inset from 0 s to 2000 s. The orange blocks highlight the time period with the α-Pinene purging in the testing chamber with the purging time: 50 ppm: 400 sec, 100 ppm: 600 sec, 500 ppm: 600 sec, 1000 ppm: 1150 sec; and 2500 ppm: 1100 sec.

### DESCRIPTION

It has been surprisingly found that a sensor with superior properties can be manufactured. Thus, in a first aspect of the invention there is provided a method of manufacturing a non-polar volatile organic chemical sensor, the method comprising the steps of:
(a) providing a composite material comprising a substrate coated by an activated Ti₃C₂ MXene layer, which activated Ti₃C₂ MXene layer has a surface; and
(b) forming a hydrophobic silane layer on the surface of the activated Ti₃C₂ MXene layer to provide the non-polar volatile organic chemical sensor, wherein
   the hydrophobic silane layer is formed by gaseous phase silanization using a monomeric hydrophobic silane compound.

This sensor has been shown to be particularly useful in the detection of VOCs derived from plants. For example, the sensors manufactured by the process outlined above may be able to detect non-polar VOC analytes down to 50 ppm.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As will be appreciated, MXenes are 2-dimensional materials, one of which is Ti₃C₂. When used herein, a “Ti₃C₂ MXene layer” refers to a layer on a substrate that comprises (or consists of) Ti₃C₂ MXene compounds.

When used herein, the term “activated Ti₃C₂ MXene layer” refers to the surface of the Ti₃C₂ MXene layer where the surface has been chemically activated in any suitable way. Such surface activations may include plasma activation (e.g. with any suitable plasma, such as oxygen plasma, and the like).

When used herein, the term “hydrophobic” refers to a material that, when provided as an outer layer (i.e. as a surface), has a water contact angle of greater than 90°.

Sensors made using Ti₃C₂ MXene have not been particularly good at sensing non-polar VOCs, such as toluene and plant VOCs (e.g. terpenes). In the current invention the hydrophilic Ti₃C₂ MXene surface is coated with a hydrophobic silane (e.g. an alkyl silane, such as trimethoxy(propyl)silane (TMPS)) which replaces (or caps) the hydroxyl groups of Ti₃C₂ MXene by a hydrophobic group (e.g. a carbon chain). Without wishing to be bound by theory, it is believed that the existence of the hydrophobic groups (e.g. carbon chain) can improve the affinity of the non-polar VOCs towards the Ti₃C₂ MXene, thereby increasing the sensing response of the non-polar VOCs.

As noted above, the hydrophobic silane layer on the surface of the activated Ti₃C₂ MXene layer may be formed using a monomeric hydrophobic silane compound. Any suitable monomeric hydrophobic silane compound (or combination thereof) may be used herein. For example, the monomeric hydrophobic silane compound may include, but is not limited to, (3-aminopropyl)trimethoxysilane (APTMS), trimethoxy(octyl)silane (TEOS), trimethoxy(propyl)silane, trimethoxymethylsilane (TEMS), and combinations thereof. In particular embodiments that may be mentioned herein, the monomeric hydrophobic silane compound may be trimethoxy(propyl)silane (TMPS).

The hydrophobic silane layer on the surface of the activated Ti₃C₂ MXene layer may have any suitable level of thickness, provided that it still allows some form of interaction between the material to be sensed and the Ti₃C₂ MXene layer. For example, the hydrophobic silane layer may have a thickness of from 10 to 14 nm.

In order for the monomeric hydrophobic silane compound to form the hydrophobic silane layer on the surface of the activated Ti₃C₂ MXene layer, it needs to undergo a gaseous phase silanization process. This process may be conducted at any suitable temperature (e.g. a temperature at which the silane(s) used are in a gaseous form). For example, the gaseous phase silanization may be conducted at a temperature of from 80 to 150° C., such as about 100° C. In additional or alternative embodiments, the gaseous phase silanization process may be conducted under reduced pressure, thereby lowering the temperature at which the monomeric hydrophobic silane compound(s) become gaseous. For example, the pressure for the gaseous phase silanization process may be from 0.1 to 0.3 mbar, such as about 0.16 mbar.

The gaseous phase silanization may make use of any suitable methodology to supply the monomeric hydrophobic silane compound(s) to the activated Ti₃C₂ MXene layer. For example, the monomeric hydrophobic silane compound(s) may be transported in a suitable carrier gas to the activated Ti₃C₂ MXene layer. For example, the gaseous phase silanization may use a flow of an inert gas for this purpose. The inert gas (e.g. argon), may have any suitable flow rate, such as from 50 to 100 sccm, such as about 65 sccm.

While the method described above provides a sensor material, this material's suitability can be enhanced further by the inclusion of one or more electrodes. Thus, in embodiments of the invention, the composite material provided in step (a) of the process above may further comprise at least two electrodes laid on the surface of the activated Ti₃C₂ MXene layer. As will be appreciated, in embodiments where the electrodes are present, they may be laid onto the surface of the activated Ti₃C₂ MXene layer before that layer is coated by the hydrophobic silane layer.

The electrodes may be formed by any suitable means on the surface of the activated Ti₃C₂ MXene layer. For example, the electrodes may be formed by thermal evaporation of an electrode material onto the surface of the activated Ti₃C₂ MXene layer. The electrode material may be any suitable conductive material, such as a metal and/or a conductive form of carbon. In particular embodiments of the invention, the electrode material may be gold.

The at least two electrodes may have any suitable thickness that allows them to function as part of the sensor. For example, the at least two electrodes may each have a thickness of from 75 to 150 nm, such as about 100 nm.

As noted hereinbefore, the activated Ti₃C₂ MXene layer may be formed by any suitable surface activation process. In embodiments of the invention that may be mentioned herein, the activated Ti₃C₂ MXene layer may be formed by the steps of:
(ai) providing a composite material comprising a substrate coated by an unactivated Ti₃C₂ MXene layer; and
(aii) activating the Ti₃C₂ MXene layer through a surface activation process.

The surface activation may be any suitable surface activation process. An example of a suitable surface activation process is plasma activation. In embodiments of the invention that may be mentioned herein, the surface activation process may be O₂ plasma activation.

As will be appreciated, the method disclosed above provided a sensor. Thus, in a further aspect of the invention, there is provided a non-polar volatile organic chemical sensor comprising:
a substrate having a substrate surface;
a Ti₃C₂ MXene layer coated on the substrate surface which Ti₃C₂ MXene layer has a Ti₃C₂ MXene layer surface;
at least two electrodes on the Ti₃C₂ MXene layer surface; and
a hydrophobic silane layer on the Ti₃C₂ MXene layer surface, wherein the hydrophobic silane layer has a thickness of from 10 to 14 nm.

The components of the sensor are as described hereinbefore, so will not be repeated again for the sake of brevity.

As will be appreciated, the hydrophobic silane layer on the Ti₃C₂ MXene layer may be formed in the manner described above. Thus, in embodiments of the invention that may be mentioned herein, the hydrophobic silane layer may be formed from a monomeric hydrophobic silane compound that is selected from one or more of the group consisting of (3-aminopropyl)trimethoxysilane (APTMS), trimethoxy(octyl)silane (TEOS), trimethoxy(propyl)silane, and trimethoxymethylsilane (TEMS). For example, the monomeric hydrophobic silane compound may be trimethoxy(propyl)silane.

The non-polar volatile organic chemical sensor described herein may be capable of detecting an analyte present in an environment at a concentration of about 50 ppm (and higher). This is advantageous, as it enables the sensor to detect concentrations that are much lower than previously thought possible. As shown in the examples below, the resultant sensing responses are higher than that of pristine MXene (without silanization) even in the range of 10-40 sccm when tested with α-Pinene (a non-polar VOC).

As will be appreciated, the sensor disclosed herein may be used to detect the presence or absence of a suitable non-polar VOC, such as toluene or a terpene (e.g. α-pinene). Thus, in a further aspect of the invention, there is provided a method of detecting an analyte comprising the steps of:
(bi) exposing a non-polar volatile organic chemical sensor as described hereinbefore to an environment where the analyte is suspected to be present for a period of time; and
(bii) subsequently measuring the resistance of the volatile organic chemical sensor to determine the presence or absence of the analyte in said environment.

As noted above, the analyte may be a volatile organic compound. More particularly, the volatile organic compound may be a non-polar volative organic compound. Examples of non-polar volatile organic compounds that may be mentioned herein includes, but are not limited to, α-pinene, 1-hexanol, a terpinol, phenethyl alcohol, and combinations thereof. In particular embodiments that may be mentioned herein, the volatile organic compound may be α-pinene. As mentioned above, the non-polar volatile organic chemical sensor may be capable of detecting an analyte present in the environment at a concentration of about 50 ppm (and higher).

Further aspects and embodiments of the invention will not be discussed by reference to the following non-limiting examples.

### EXAMPLES

### Materials

TisAlC₂ (≥98%) was purchased from Famouschem Technology Co., Ltd. LiF powder (powder, <100 μm, ≥99.98% trace metals basis) was purchased from Sigma-Aldrich.

### Example 1. Fabrication of TMPS-Modified Ti₃C₂ MXene VOC Sensor

Preparation of Ti₃C₂ MXene Nanoflakes

1 g of TisAlC₂ was mixed with 20˜30 mL of HCl with concentration between 9 M to 12 M for 24 h at 25° C. The mixture was then washed with deionized (DI) water by centrifugation for at least 4 times at 10000 rpm for 5 min. The sediment was collected and stored at −20° C. to reduce the dissolved oxygen. The frozen MXene sediment was then dispersed in DI water (300 mL to 500 mL) by bath sonication for 1 h with Ar gas bubbling. The MXene dispersion was subjected to centrifugation at 3000 rpm. The supernatant was then collected and stored at 4° C. for further use.

Deposition Method of Ti₃C₂ MXene Thin Film

The deposition of the Ti₃C₂ MXene was carried out by spin casting. At first, the intended substrate, glass slide, was washed by ultrasonication in acetone for 30 min. After ultrasonication, the glass slide was dried by Ar gas with an air blowing gun. The dried glass slide was then subjected to oxygen (O₂) plasma treatment to create the hydrophilic surface. The parameters for the oxygen plasma treatment were set following this procedure: pumping down the chamber to 9.00 E⁻² torr, applying the oxygen gas with the flow rate at 30 sccm for 1 min, and setting the RF power at 100 W and the RF frequency at 50 KHz.

After O₂ plasma treatment, the glass slide was directly used for spinning casting of Ti₃C₂ MXene suspension. The Ti₃C₂ MXene suspension (0.8 mg/ml to 1.5 mg/mL) was casted before the spinning the sample stage. Normally, around 400 μL of Ti₃C₂ MXene liquid was needed to fully cover the glass slide (2.5 mm×2.5 mm). The spin rate of the sample stage was programmed to have two spinning steps: first step for MXene deposition and the final step for drying of the glass slide. The spin rate of the stage was set to be 1000 rpm for 60 s and the spin rate was then ramped to 500 rpm to 2000 rpm for another 60 s for sample drying. The substrate deposited with Ti₃C₂ MXene was then dried under vacuum at room temperature (23.5° C. to 25° C.) for 24 h. After drying, the sample was taken for electrode deposition via thermal evaporation.

Fabrication of Ti₃C₂ MXene VOC Sensor

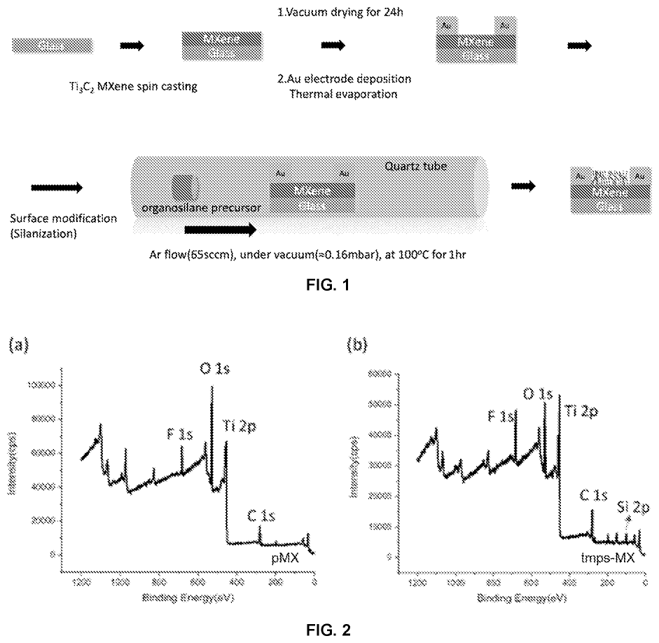

The fabrication of Ti₃C₂ MXene VOC sensor was carried out as shown in FIG. 1. After the Ti₃C₂ thin film coated glass slide prepared above was dried for 24 h at room temperature, the sample was taken for gold (Au) electrode deposition. The deposition of the Au electrode was conducted by thermal evaporation under high vacuum at the pressure below 3×10⁻⁵ torr. As the pressure pumped down below 3×10⁻⁵ torr, the Au pellet fed tungsten (W) boat was heated by joule heating. The rate of Au deposition can be controlled by varying the current applied to the W boat. At first, the rate of the Au deposition was fixed at 0.1 Å/s for the first 10 nm Au layer in order to obtain better adhesion between Au and MXene layer. Later, the rate of the Au deposition was increased to 1 Å/s and the deposition was terminated when the thickness of the Au layer was 100 nm. The deposition time was about 15-20 minutes until the final thickness was 100 nm. The rate of evaporation was 1 Å/s. The as-prepared Ti₃C₂ MXene VOC sensor was immediately taken for MXene surface modification after the sample was removed from the evaporation chamber.

Surface Modification of Ti₃C₂ MXene VOC Sensor

The surface modification of the Ti₃C₂ MXene thin film on the Ti₃C₂ MXene VOC sensor prepared above was conducted by gaseous phase silanization in a tube furnace. The Au electrode-deposited Ti₃C₂ MXene-coated glass slide was placed in the middle zone of the tube furnace and 1 mL of organosilane (TMPS) was placed at the upstream side of the quartz tube around 25 cm away from the glass slide (FIG. 1). The alkyl silane replaces the hydroxyl groups of Ti₃C₂ MXene with carbon chain. During the reaction, the heating temperature was set to 100° C. with the ramping rate of temperature at 5° C./min and the flow rate of the carrier gas, Ar gas, was fixed at 65 sccm. The reaction was conducted under vacuum with the pressure of around 0.16 mbar for 1 h at 100° C. to give the TMPS-modified Ti₃C₂ MXene.

### Example 2. Characterization of TMPS-Modified Ti₃C₂ MXene VOC Sensor

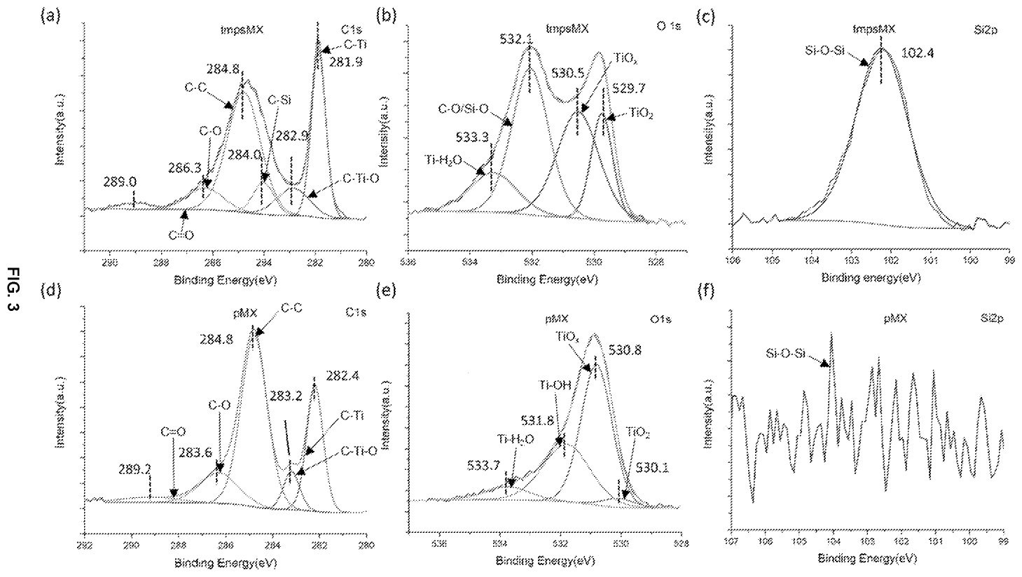

The evidence that the TMPS was modified on the surface of Ti₃C₂ MXene was verified by XPS as shown in FIG. 2. The Si 2p peak is found at around 100 eV in TMPS-modified Ti₃C₂ MXene (FIG. 2), indicating the chemical binding between TMPS and Ti₃C₂ MXene. To further investigate the chemical composition and the binding information after Ti₃C₂ MXene was modified with TMPS, narrow range scanning of XPS was performed. The TMPS-modified Ti₃C₂ MXene presents six deconvoluted peaks at, 281.9 eV, 282.9 eV, 284.0 eV, 284.8 eV, 286.3 eV and 289.0 eV, which can be assigned to the binding of Ti—C, C—Ti—O, C—Si, C—C, C—O, and C═O respectively (FIG. 3(a)). As compared to the spectrum of TMPS-modified Ti₃C₂ MXene (FIG. 3(a)), the pristine Ti₃C₂ MXene shows less deconvoluted peaks with the binding energy centered at 282.4 eV, 283.2 eV, 284.8 eV, 283.6 eV and 289.2 eV, which can be assigned to the binding of C—Ti, C—Ti—O, C—C, C—O, and C═O (FIG. 3(d)). Since the pristine Ti₃C₂ MXene was treated with Ar plasma to remove the surface oxide and contamination, some of the termination group on the C—Ti bonding was also removed, leading to higher measured Ti—C binding energy at 282.4 eV as compared to the TMPS-modified Ti₃C₂ MXene at 281.9 eV (J. Halim et al., Appl. Surf. Sci. 2016, 362, 406-417; S. Shah et al., Chem. Commun. 2017, 53, 400-403; and W. Y. Chen et al., ACS Nano 2020, 14, 11490-11501). Also, the formation of C—Si binding is found at 282.9 eV after surface silanization with TMPS, which suggests that the TMPS molecules are covalently bond to the MXene surface.

### Example 3. Resistance of Pristine Ti₃C₂ MXene and TMPS-Modified Ti₃C₂ MXene

The resistance of the Ti₃C₂ MXene thin film and TMPS-modified Ti₃C₂ MXene was measured by the two-terminal method.

### Two-Terminal Method

To test the conductivity of the sensor, the sensor was subjected to two terminal I-V test under pure N₂ exposure. The purging conditions were until the resistance stabilized and there was no or very little change in resistance observed when it was exposed to nitrogen. Nitrogen was purged at 200 sccm flow rate during this time. The sensor typically has a linear I-V characteristic for an applied voltage between −1V to 1V.

### Results and Discussion

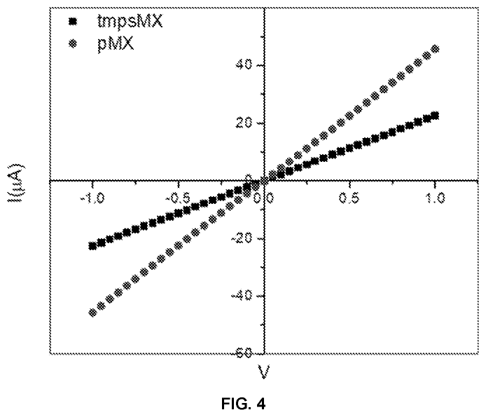

The linear I-V curve shows the ohmic behavior and indicates the stable conductance of the pristine and TMPS-modified Ti₃C₂ MXene thin film (FIG. 4). The resistance of the pristine and TMPS-modified Ti₃C₂ MXene was then calculated by ohm's law, showing the resistance of pristine Ti₃C₂ MXene at 22.03 kΩ and TMPS-modified Ti₃C₂ MXene at 44.35 kΩ.

### Example 4. VOC Sensing Performance of Pristine Ti₃C₂ MXene and TMPS-Modified Ti₃C₂ MXene

The existence of the carbon chain can improve the affinity of the non-polar VOCs towards Ti₃C₂ MXene, thereby increasing the sensing response of the non-polar VOCs.

### VOC Sensing Performance Test

To measure the gas sensing response of the sensor (pristine Ti₃C₂ MXene or TMPS-modified Ti₃C₂ MXene), the sensor was placed in a gas sensing chamber with an inlet and an outlet of gas channel. In addition, a simple probe station was installed with the gas sensing system to obtain the electrical signal from the MXene sensors.

The generation of the VOC vapors was carried out by the bubbling of carrier gas (N₂) through the pure VOC chemicals. The concentration of the VOC can be controlled by the flow rate of N₂ blowing through the bubbler and the dilution gas (N₂) during gas mixing. The total flow rate of the N₂ was fixed at 200 sccm during the measurement. The concentration of the VOC was calculated following the equation (1):

$$Concentration ( ppm ) = ( \frac{{P}_{s}}{P} \times \frac{f}{f + F} ) \times {10}^{6} \qquad ( 1 )$$

Where Pₛ refers to the saturated partial pressure in mm-Hg and P refers to the total pressure; f and F refer to the flow rate of the VOC line and the dilution line, respectively. The saturated partial pressure of VOC can be calculated from Antoine equation (2):

$$Log {P}_{s} = ( A - \frac{B}{C + T} ) \qquad ( 2 )$$

Where A, B and C are the Antoine coefficients and T is the temperature in Kelvin.

To investigate the VOC sensing performance, TMPS-modified Ti₃C₂ MXene and pristine Ti₃C₂ MXene were exposed to α-Pinene (a terpene and non-polar plant VOC) with various concentration from 50 ppm to 2500 ppm (50 ppm, 100 ppm, 1000 ppm or 2500 ppm). The sensing of α-Pinene can be observed by the change in the resistance as the pristine and TMPS-modified Ti₃C₂ MXene were applied with a constant DC voltage. The sensing response was calculated following the equation (3):

$$Gas sensing response ( \% ) = Δ R / {R}_{0} \times 100 = ( {R}_{g} - {R}_{0} ) / {R}_{0} \times 100 \qquad ( 3 )$$

Where R₀ and R_{g} refer to the resistance of the sensor in the N₂ and exposed to the analytes.

### Results and Discussion

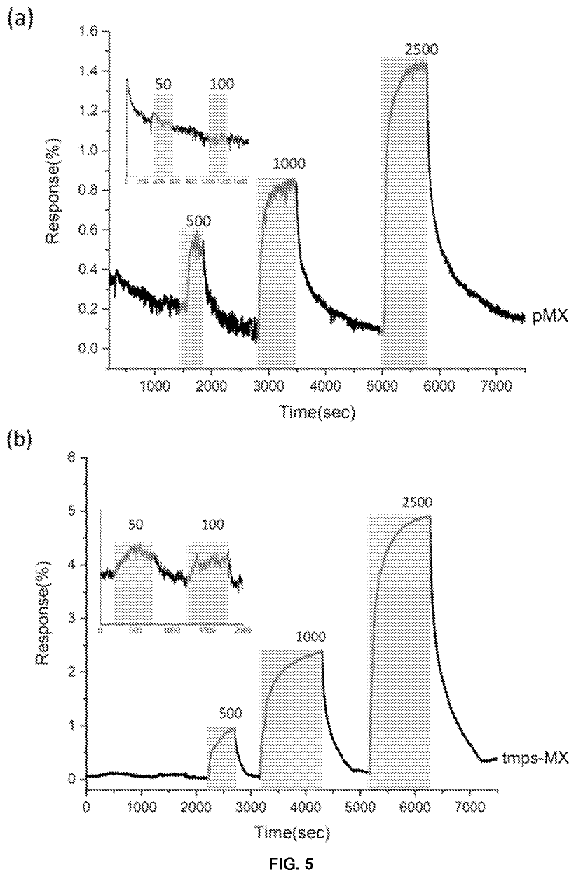

From FIG. 5 and Table 1, the TMPS-modified Ti₃C₂ MXene showed improved sensing response towards α-Pinene at 0.06%, 0.04%, 0.93%, 2.36%, 4.78% for 50 ppm, 100 ppm, 1000 ppm, and 2500 ppm, respectively, as compared to pristine Ti₃C₂ MXene. For instance, the sensing response of α-Pinene in TMPS-modified Ti₃C₂ MXene was 2.36% at 1000 ppm, whereas the sensing response detected by pristine Ti₃C₂ MXene was 0.74%. Additionally, the TMPS-modified Ti₃C₂ MXene showed a signal even at the lower limit of the detection range at 50 ppm and below, while the pristine Ti₃C₂ MXene did not show obvious signal under such low concentration of α-Pinene, which exhibits the improved sensing specificity of non-polar VOC by Ti₃C₂ MXene-based VOC sensor.

TABLE 1
The summarized gas sensing response of α-Pinene
by pristine and TMPS-modified Ti₃C₂ MXene.

| Concentration | 2500 ppm | 1000 ppm | 500 ppm | 100 ppm | 50 ppm |
|---|---|---|---|---|---|
| pMX | 1.33% | 0.74% | 0.5% | N/A | N/A |
| tmpsMX | 4.78% | 2.36% | 0.93% | 0.04% | 0.06% |

N/A: data not available due to unidentifiable signal to noise ratio.

1. A method of manufacturing a non-polar volatile organic chemical sensor, the method comprising the steps of:
(a) providing a composite material comprising a substrate coated by an activated Ti₃C₂ MXene layer, which activated Ti₃C₂ MXene layer has a surface; and
(b) forming a hydrophobic silane layer on the surface of the activated Ti₃C₂ MXene layer to provide the non-polar volatile organic chemical sensor, wherein
the hydrophobic silane layer is formed by gaseous phase silanization using a monomeric hydrophobic silane compound.

2. The method according to claim 1, wherein the monomeric hydrophobic silane compound is selected from one or more of the group consisting of (3-aminopropyl)trimethoxysilane (APTMS), trimethoxy(octyl)silane (TEOS), trimethoxy(propyl)silane, and trimethoxymethylsilane (TEMS).

3. The method according to claim 2, wherein the monomeric hydrophobic silane compound is trimethoxy(propyl)silane.

4. The method according to claim 1, wherein the hydrophobic silane layer has a thickness of from 10 to 14 nm.

5. The method according to claim 1, wherein the gaseous phase silanization is conducted at from 80 to 150° C. and a pressure of from 0.1 to 0.3 mbar.

6. The method according to claim 1, wherein the gaseous phase silanization used a flow of an inert gas at a flow rate of from 50 to 100 sccm.

7. The method according to claim 1, wherein the composite material provided in step (a) of claim 1 further comprises at least two electrodes laid on the surface of the activated Ti₃C₂ MXene layer.

8. The method according to claim 7, wherein the electrodes are formed by thermal evaporation of an electrode material onto the surface of the activated Ti₃C₂ MXene layer.

9. The method according to claim 1, wherein the activated Ti₃C₂ MXene layer is formed by the steps of:
(ai) providing a composite material comprising a substrate coated by an unactivated Ti₃C₂ MXene layer; and
(aii) activating the Ti₃C₂ MXene layer through a surface activation process.

10. The method according to claim 9, wherein the surface activation process is plasma activation.

11. A non-polar volatile organic chemical sensor comprising:
a substrate having a substrate surface;
a Ti₃C₂ MXene layer coated on the substrate surface which Ti₃C₂ MXene layer has a Ti₃C₂ MXene layer surface;
at least two electrodes on the Ti₃C₂ MXene layer surface; and
a hydrophobic silane layer on the Ti₃C₂ MXene layer surface, wherein the hydrophobic silane layer has a thickness of from 10 to 14 nm.

12. The sensor according to claim 11, wherein the hydrophobic silane layer is formed from a monomeric hydrophobic silane compound that is selected from one or more of the group consisting of (3-aminopropyl)trimethoxysilane (APTMS), trimethoxy(octyl)silane (TEOS), trimethoxy(propyl)silane, and trimethoxymethylsilane (TEMS).

13. The sensor according to claim 12, wherein the monomeric hydrophobic silane compound is trimethoxy(propyl)silane (TMPS).

14. The sensor according to claim 11, wherein:
(I) the electrode material is gold; and/or
(II) the at least two electrodes each have a thickness of from 75 to 150 nm, such as about 100 nm.

15. The sensor according to claim 11, wherein the non-polar volatile organic chemical sensor is capable of detecting an analyte present in an environment in a concentration of about 50 ppm.

16. A method of detecting an analyte comprising the steps of:
(bi) exposing a non-polar volatile organic chemical sensor as described in claim 11 to an environment where the analyte is suspected to be present for a period of time; and
(bii) subsequently measuring the resistance of the volatile organic chemical sensor to determine the presence or absence of the analyte in said environment.

17. The method according to claim 16, wherein the analyte is a volatile organic compound.

18. The method according to claim 17, wherein the volatile organic compound is selected from one or more of α-pinene, 1-hexanol, a terpinol, and phenethyl alcohol.

19. The method according to claim 16 wherein the non-polar volatile organic chemical sensor is capable of detecting an analyte present in the environment in a concentration of about 50 ppm.
